(19) United States
(12) Patent Application Publication
(10) Pub. No.: US 2022/0183940 A1
(43) Pub. Date: Jun. 16, 2022

(54) DYEING PROCESS USING PEROXYGENATED SALTS AND A SUBSTRATE COMPRISING OXIDATION DYES

(71) Applicant: L'OREAL, Paris (FR)

(72) Inventors: Leila Hercouet, Saint Ouen (FR); Simon Donck, Saint Ouen (FR); Henri Samain, Chevilly-Larue (FR)

(73) Assignee: L'OREAL, Paris (FR)

(21) Appl. No.: 17/599,440

(22) PCT Filed: Mar. 27, 2020
(86) PCT No.: PCT/EP2020/058856

§ 371 Date: Sep. 28, 2021

(30) Foreign Application Priority Data
Mar. 29, 2019 (FR) FR1903399

(51) Int. Cl. A61K 8/23; A61Q 5/10; A61K 8/02

(52) U.S. Cl. CPC A61K 8/23; A61Q 5/10; A61K 2800/87; A61K 2800/43; A61K 2800/884; A61K 8/0204

(57) ABSTRACT

The present invention relates to a process for dyeing keratin fibres, in particular human keratin fibres such as the hair, comprising the successive application to said fibres (i) of a composition (A) containing at least one peroxygenated salt and (ii) of a substrate including a surface totally or partially coated with at least one layer containing one or more oxidation dyes; said process does not use any hydrogen peroxide during the application of the substrate to the fibres.

The present invention relates to the field of dyeing keratin fibres and more particularly to the field of hair dyeing.

The present invention notably relates to a process for dyeing keratin fibres, in particular human keratin fibres such as the hair, comprising the successive application to said fibres (i) of a composition (A) containing at least one peroxygenated salt and (ii) of a substrate including a surface totally or partially coated with at least one layer containing one or more oxidation dyes; said process not using any hydrogen peroxide during the application of the substrate to the fibres.

Conventional oxidation dyeing processes generally consist in applying to keratin fibres a dye composition comprising oxidation bases and couplers with hydrogen peroxide (H₂O₂ or aqueous hydrogen peroxide solution), as oxidizing agent, in leaving it to diffuse, and then in rinsing said fibres. The colourings resulting therefrom are generally permanent, strong and resistant to external agents, notably to light, bad weather, washing, perspiration and rubbing.

However, it has been found that it is often difficult or even virtually impossible to obtain colourings with novel visual results by means of conventional oxidation dyeing techniques. In particular, such processes do not satisfactorily afford coloured or multi-coloured patterns, which may lead to novel, appealing optical effects on the entire head of hair, which prove to be both sharp and precise.

These conventional oxidation dyeing processes also have the drawback of staining the hands of the user or of the hair stylist during the application to the hair of the ready-to-use composition resulting from mixing the dye composition and the oxidizing composition. Similarly, this type of process may also give rise to undesirable stains on the user's scalp, contour of the face and/or clothing, which may be due to application errors and/or to problems of running of the compositions.

These conventional oxidation dyeing processes also entail the risk of not leading to the final colouring desired by the user because of an error arising during the handling of the dye compositions and oxidizing compositions or because of a poor choice of the starting dye compositions.

It has also been found that the storage of the dye compositions and oxidizing compositions used for obtaining the desired colouring may pose problems of space occupation, notably in hairstyling salons.

These oxidation dyeing processes may thus prove to be impractical for achieving many varied colours as a function of the different users.

In order to overcome these various drawbacks, it has already been proposed in FR 3 015 895 to perform a dyeing process consisting in placing the keratin fibres in contact with a substrate which is in the form of a sheet, which has been pretreated with a composition comprising one or more oxidation dyes, and then with an aqueous oxidizing composition. By means of this pretreatment, the oxidation dyes form a layer which partly or totally coats the surface of the substrate. The oxidation dyes may thus be deposited onto the surface of the substrate uniformly or non-uniformly and may adopt one or more novel geometrical shapes in order to produce different types of coloured patterns.

In particular, the keratin fibres are placed on the substrate and the oxidizing composition is then applied to said fibres and/or the substrate. In the course of the dyeing process, the oxidation dyes are extracted from the surface of the substrate so as to migrate towards the keratin fibres to penetrate them and will react with the oxidizing agent(s) of the oxidizing composition so as to dye the fibres.

Such a process has the advantage of leading to persistent colourings which may be uniform, multi-coloured and/or bearing coloured patterns so as to produce, for example, shading-off, spots, novel geometrical shapes and/or reproductions of images on the keratin fibres. In particular, such a process makes it possible to create visually novel coloured patterns that can be personalized by the consumer.

However, such a process involves leave-on times that may prove to be rather long, generally of the order of about 30 minutes, and which are notably due to the fact that the oxidation dyes must first be extracted from the substrate before dyeing the keratin fibres.

In order to shorten this leave-on time, it has been envisaged to modify the very nature of the substrate so as to promote faster extraction of the dyes so that they can react with the oxidizing agent(s) of the oxidizing composition and access the keratin fibres more easily.

To this end, it has already been proposed to use substrates which do not absorb oxidation dyes, notably using plasticized sheets.

However, the leave-on times observed for this type of process still too often remain long, generally of the order of 25 minutes.

Furthermore, this type of substrate is problematic as regards large-scale production. Specifically, during the pretreatment of the substrate with the composition containing the oxidation dye(s), said composition may be incorrectly deposited and may produce pools on the surface of the substrate, which has a negative impact on the sharpness of the coloured patterns.

There is thus a real need to perform a process for dyeing keratin fibres, in particular human keratin fibres such as the hair, which does not have the drawbacks mentioned previously, i.e. which is notably capable of leading to sharp and precise coloured patterns rapidly and simply while at the same time reducing the space occupation problems and minimizing the risks of contact that may arise between the compositions used and the user's hands, scalp and/or clothing.

This aim is achieved by the present invention, one subject of which is notably a process for dyeing keratin fibres, in particular human keratin fibres such as the hair, by successively applying to said fibres:

(i) a composition (A) containing one or more peroxygenated salts;

(ii) at least one substrate including a surface coated with at least one layer containing one or more oxidation dyes;

given that the process does not use any hydrogen peroxide during the application of the substrate to the fibres.

The dyeing process according to the invention thus makes it possible rapidly and simply, i.e. with short leave-on times, to produce sharp and precise coloured patterns on keratin fibres, thus giving them a novel and appealing aesthetic appearance.

In particular, the dyeing process according to the invention makes it possible to achieve such results with shorter leave-on times than those observed with the previous dyeing processes.

In other words, the leave-on times involved in the process according to the invention are advantageously shortened.

In particular, the leave-on time of the substrate may advantageously range from 5 to 10 minutes, which is barely longer than the leave-on time of composition (A).

The dyeing process according to the invention thus uses at least one substrate including a surface coated with at least one layer containing one or more oxidation dyes. In other words, the substrate has been pretreated on its surface with a dye preparation composition containing one or more oxidation dyes.

The substrate has the advantage of being able to be easily stored in the user's home when compared with the use of dye compositions used in conventional oxidation dyeing processes, thereby making it possible to substantially reduce the space occupation.

The substrate may advantageously be prepared directly in the hairstyling salon or beforehand.

The dyeing process according to the invention thus makes it possible to produce on the keratin fibres, with great precision, coloured patterns that are visually sharp. More particularly, the process makes it possible to produce millimetre-sized coloured patterns having all types of shapes, such as spots or waves, which are readily reproducible. These patterns may thus lead to novel optical effects when they are then repeated over the entire head of hair.

In other words, the dyeing process according to the invention makes it possible to obtain patterns, notably millimetre-sized patterns, homogeneously over the entire head of hair, or in a localized manner on a part of the head of hair. These patterns may be imaginative from an aesthetic viewpoint or may serve to hide an irregularity in the colour or appearance of the keratin fibres, notably in the case of hair regrowth or fading of the ends.

Moreover, by using substrates comprising one or more oxidation dyes, i.e. substrates that have been pretreated with a composition containing such dyes, this process makes it possible to reduce the risks of staining on the user's hands, scalp, face and/or clothing. Specifically, this process makes it possible to more easily avoid the problems of running and/or errors in application of the dye compositions and oxidizing compositions.

The dyeing process according to the invention also makes it possible to obtain colourings and/or patterns whose colourings are powerful and persistent with respect to external agents (such as shampooing, light, perspiration or bad weather).

In particular, the dyeing process according to the invention leads to the production of patterns whose colouring is powerful and resistant with respect to shampooing.

Furthermore, the colour build-up brought about by the process according to the invention is high.

In accordance with the present invention, the process does not use any hydrogen peroxide during the application of the substrate to the keratin fibres.

In other words, a composition containing hydrogen peroxide is not added when the substrate is applied to the keratin fibres, i.e. when the substrate comes into contact with said fibres.

In other words, the process according to the invention does not comprise the application to the keratin fibres of a composition comprising hydrogen peroxide during the application of the substrate to the keratin fibres.

In still other words, the process of the present invention does not comprise a step of applying to the keratin fibres a composition comprising hydrogen peroxide when the substrate as previously defined is applied to said fibres.

In particular, the step of applying the substrate to the keratin fibres does not involve the implementation of a composition containing hydrogen peroxide.

More particularly, hydrogen peroxide is not applied to the keratin fibres when the substrate is applied to the keratin fibres.

According to an embodiment of the process, hydrogen peroxide may be implemented during said process before the substrate is applied to the keratin fibres.

Other subjects, characteristics, aspects and advantages of the invention will emerge even more clearly on reading the description and the examples that follow.

For the purposes of the present invention and unless otherwise indicated:
a “heteroaryl radical” represents an optionally cationic, 5- to 22-membered monocyclic or fused or non-fused polycyclic group, comprising from 1 to 6 heteroatoms chosen from nitrogen, oxygen, sulfur and selenium, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl, xanthyl and thioxanthinyl;
an “aryl” radical represents a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl;
the “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl or thiol group;
   a C₁-C₆ alkoxy or C₁-C₆ alkylthio radical;
   a (poly)hydroxy(C₂-C₆)alkoxy radical;
   an amino radical;
   a 5- or 6-membered heterocycloalkyl radical, preferentially morpholino, piperazino, piperidino or pyrrolidino, which is optionally substituted with a (C₁-C₄)alkyl radical, preferentially methyl;
   a 5- or 6-membered heteroaryl radical, preferentially imidazolyl, optionally substituted with a (C₁-C₄)alkyl radical, preferentially methyl;
   an amino radical substituted with one or two identical or different C₁-C₆ alkyl radicals, optionally bearing at least:
a hydroxyl group,
an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
a quaternary ammonium group —N⁺R′R″R′″, M⁻ for which R′, R″ and R′″, which may be identical or different, represent a hydrogen atom or a C₁-C₄ alkyl group and M⁻ represents an anionic counterion,
or an optionally cationic 5- or 6-membered heteroaryl radical, preferentially imidazolium, optionally substituted with a (C₁-C₄)alkyl radical, preferentially methyl;
   an acylamino radical (—N(R)—C(O)—R′) in which the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
a cyclic or heterocyclic radical, or a non-aromatic part of an aryl or heteroaryl radical, may also be substituted with one or more oxo groups;
an “alkyl radical” is a linear or branched C₁-C₁₀, in particular C₁-C₈, more particularly C₁-C₆ and preferably C₁-C₄ hydrocarbon-based radical;
the limits of a range of values are included in that range, notably in the expressions “between . . . and . . . ” and “ranging from . . . to . . . ”;
the expression “at least one” is equivalent to the expression “one or more” and may be replaced therewith.

First Step—Composition (A)

As indicated previously, the dyeing process uses on said fibres a composition (A) containing one or more peroxygenated salts as chemical oxidizing agent.

Composition (A) is used on the keratin fibres, i.e. it may be applied to the keratin fibres or it may be mixed with a composition comprising one or more chemical oxidizing agents, preferably hydrogen peroxide, and the resulting mixture is then applied to said fibres.

In other words, composition (A) may be applied to the keratin fibres or composition (A), after mixing with a composition comprising one or more chemical oxidizing agents, preferably hydrogen peroxide (i.e. the composition resulting from the mixing), is applied to the keratin fibres.

The term “chemical oxidizing agent” means any chemical oxidizing agent other than atmospheric oxygen.

The peroxygenated salt(s) are notably chosen from persulfates, perborates, peracids and/or salts thereof, percarbonates, in particular of alkali metals or alkaline-earth metals, and mixtures thereof.

Preferably, the peroxygenated salt(s) are chosen from persulfates.

More preferentially, the peroxygenated salt(s) are chosen from sodium, potassium and ammonium persulfates, and mixtures thereof, in particular sodium persulfate.

The content of peroxygenated salt(s) may range from 1% to 60% by weight and preferably from 10% to 50% by weight relative to the total weight of composition (A).

Composition (A) may also comprise one or more alkaline agents.

The alkaline agents may be chosen from carbonates, alkanolamines such as monoethanolamine, diethanolamine, triethanolamine and also derivatives thereof, oxyethylenated and/or oxypropylenated ethylenediamines, mineral or organic hydroxides, alkali metal silicates such as sodium metasilicate, amino acids, preferably basic amino acids such as arginine, lysine, ornithine, citrulline and histidine, and also the compounds of formula (I) below:

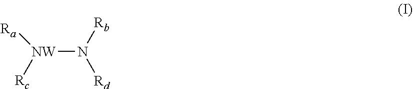

in which:
W is a divalent (C₁-C₈)alkylene group, preferably propylene optionally substituted notably with a hydroxyl group or a C₁-C₄ alkyl radical;
Rₐ, R_{b}, R_{c} and R_{d}, which may be identical or different, represent a hydrogen atom or a C₁-C₄ alkyl or C₁-C₄ hydroxyalkyl radical.

The mineral or organic hydroxides are preferably chosen from i) hydroxides of an alkali metal, ii) hydroxides of an alkaline-earth metal, for instance sodium hydroxide or potassium hydroxide, iii) hydroxides of a transition metal, such as hydroxides of metals from groups III, IV, V and VI, iv) hydroxides of lanthanides or actinides, quaternary ammonium hydroxides and guanidinium hydroxide.

The hydroxide may be formed in situ, for instance guanidine hydroxide, formed by reacting calcium hydroxide with guanidine carbonate.

Preferably, composition (A) comprises one or more alkaline agents chosen from alkali metal silicates, in particular sodium metasilicate.

Composition (A) may also comprise one or more surfactants, preferably one or more nonionic and/or anionic surfactants.

Preferably, the nonionic surfactants are oxyalkylenated and chosen from oxyethylenated C₈-C₃₀ alcohols, and polyoxyethylenated esters of saturated or unsaturated, linear or branched C₈-C₃₀ acids and of sorbitol.

The anionic surfactants may be chosen from sulfate, sulfonate, carboxylic (or carboxylate) surfactants, and mixtures thereof, notably sulfate and carboxylic surfactants.

Preferably, the anionic surfactant(s) are chosen from:
C₆-C₂₄ and notably C₁₂-C₂₀ alkyl sulfates;
C₆-C₂₄ and notably C₁₂-C₂₀ alkyl ether sulfates; preferably comprising from 2 to 20 ethylene oxide units;
C₆-C₂₄ alkyl sulfosuccinates and notably C₁₂-C₂₀ alkyl sulfosuccinates; notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acylisethionates;
C₆-C₂₄ and notably C₁₂-C₂₀ acylsarcosinates; notably palmitoyl sarcosinates;
(C₆-C₂₄)alkyl ether carboxylates, preferably (C₁₂-C₂₀)alkyl ether carboxylates;
polyoxyalkylenated (C₆-C₂₄)alkyl(amido) ether carboxylic acids and salts thereof, in particular those including from 2 to 50 alkylene oxide and in particular ethylene oxide groups;
C₆-C₂₄ and notably C₁₂-C₂₀ acylglutamates;
C₆-C₂₄ and notably C₁₂-C₂₀ acylglycinates;
and mixtures thereof;
in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferably, the surfactant(s) may represent a content ranging from 0.1% to 50% by weight and preferably from 0.5% to 30% by weight relative to the total weight of composition (A).

According to one embodiment, the use of composition (A) advantageously leads to bleaching of the keratin fibres.

For the purposes of the present invention, the term “bleaching of keratin fibres” means that the application of composition (A) leads to a difference in colour of the keratin fibres ΔE, determined by a standard colorimetric machine in the CIE L*, a* b* international system, such as a Minolta CM 2002 colorimeter, of greater than or equal to 2 relative to unbleached keratin fibres.

ΔE corresponds to the difference in colour between a lock of bleached hair and a lock of unbleached hair, i.e. of hair not treated with composition (A), and is measured according to the following equation:

ΔE=\sqrt{(L*−Lₒ*)²+(a*−aₒ*)²+(b*−bₒ*)²} (i)

In this equation, L*, a* and b* represent, respectively, the values measured on the bleached lock, and L₀*, a₀* and b₀* represent, respectively, the values measured on the unbleached lock.

In this L*a*b* system, L* represents the lightness, a* indicates the green/red colour axis and b* indicates the blue/yellow colour axis. The higher the value of L, the lighter or less intense the colour. Conversely, the lower the value of L, the darker or more intense the colour. The higher the value of a*, the redder the shade, and the higher the value of b*, the yellower the shade.

In accordance with this embodiment, the content of peroxygenated salt(s) may range from 1% to 60% by weight and preferably from 10% to 50% by weight relative to the total weight of composition (A).

In accordance with this embodiment, composition (A) is preferably mixed with a composition comprising one or more chemical oxidizing agents, in particular hydrogen peroxide, and the resulting mixture is then applied to the keratin fibres. In this way, the keratin fibres are thus bleached.

In accordance with this embodiment, composition (A) comprises between 10% and 60% by weight (i.e. between 5% and 30% by weight after mixing with the chemical oxidizing agent); preferably, the pH is alkaline (between 8 and 10).

According to another embodiment, the use of composition (A) does not lead to bleaching of the keratin fibres.

For the purposes of the present invention, the term “no bleaching of keratin fibres” means that the application of composition (A) leads to lightening of the keratin fibres ΔE, determined using a standard colorimetric machine in the CIE L*, a* b* international system, such as a Minolta CM 2002 colorimeter, of strictly less than 2 relative to unbleached keratin fibres.

ΔE corresponds to the difference in colour between a lock of bleached hair and a lock of unbleached hair, i.e. of hair not treated with composition (A), and is measured according to equation (i) defined previously.

Thus, if the colour difference ΔE is strictly less than 2, this means that the colour difference between a lock of bleached hair and a lock of unbleached hair is not significantly different and that the application of composition (A) has not led to bleaching of the lock.

In accordance with this embodiment, composition (A) is notably an aqueous composition with a pH ranging from 1 to 12, preferably less than 7, and containing the peroxygenated salt(s) defined previously.

More preferentially, the pH of composition (A) ranges from 1 to 6, even more preferentially from 1.5 to 5 and better still from 2 to 5.

In accordance with this embodiment, the acidifying agents may be chosen from organic or mineral acids such as hydrochloric acid, phosphoric acid, lactic acid or citric acid, and mixtures thereof, preferably phosphoric acid.

In accordance with this embodiment, composition (A) is an aqueous composition with an acidic pH, i.e. a pH of less than 7, preferably with a pH ranging from 1.5 to 5, better still from 2 to 4, and consists of one or more peroxygenated salts, preferably chosen from persulfates, notably sodium persulfate.

Preferably, in accordance with this embodiment, composition (A) may be prepared just before use by mixing the peroxygenated salt(s) in powder form and an aqueous phase comprising water and at least one acidifying agent, preferably an aqueous phase consisting of water and at least one acidifying agent.

In accordance with this embodiment, the content of peroxygenated salt(s) may range from 0.1% to 20% by weight and preferably from 0.5% to 5% by weight relative to the total weight of the composition. The pH preferably ranges from 1.5 to 5.

Preferably, the use of composition (A) does not lead to bleaching of the keratin fibres.

Preferably, composition (A) according to this embodiment does not comprise hydrogen peroxide.

According to this preferred embodiment, composition (A) is advantageously an aqueous composition with a pH ranging from 1 to 12, preferably less than 7, and containing the peroxygenated salt(s) defined previously. Preferably, pH ranges from 1.5 to 5, better still from 2 to 4,

Preferably, peroxygenated salt(s) are chosen from persulfates, notably sodium persulfate.

Preferably, composition (A) is rinsed out or left on, preferably left on.

More preferably, the process comprises a rinsing step between (i) the application of composition (A) and (ii) the application of the substrate.

In particular, the process comprises rinsing of the keratin fibres on conclusion of the leave-on time of composition (A).

Second Step—Substrate

As indicated previously, the dyeing process uses on said fibres at least one substrate including a surface coated with at least one layer containing one or more oxidation dyes.

The substrate is used after the keratin fibres are placed in contact with composition (A) as defined previously.

The substrate may be in the form of an element in sheet form or in another embodiment.

Preferably, the substrate is an element in sheet form.

The element in sheet form may be made of plastic material, in particular thermoplastic, paper, metal, notably aluminium, a woven, a nonwoven of non-absorbent fibres, notably of cellulose or a derivative thereof, or polyamide 6,6.

Preferably, the element in sheet form is a sheet of plastic material, notably of thermoplastic, or a nonwoven material of non-absorbent fibres, notably a nonwoven based on cellulose or a derivative thereof.

In particular, the element in sheet form is a sheet of nonwoven material of non-absorbent fibres, notably a nonwoven based on cellulose or a derivative thereof.

More particularly, the element in sheet form may be a paper of kraft type, which has the advantage of printing well and of leading to precise patterns. Specifically, the coloured patterns obtained on the keratin fibres do not run after being placed in contact with composition (A).

In particular, the element in sheet form used in the dyeing process is a plastic sheet.

In this case, the element in sheet form has the advantage of satisfactorily rendering the colouring power, which makes it possible to lead to patterns whose colouring is powerful. Furthermore, the sheet of plastic material does not absorb the water that may be present in composition (A), which makes it possible to avoid creating dry areas under the keratin fibres during the application of said composition.

The element in sheet form may consist of a water-soluble material, which makes it possible, for example, to remove it by washing the hair.

The element in sheet form preferably includes an assembly of a layer of a water-soluble material and a layer of a water-insoluble material, for example an aluminium foil.

The substrate may be designed to be able to be closed around a lock of hair. In this case, such a substrate is, for example, provided with a fastening means for keeping it in such a state, for example an adhesive positioned close to one edge or a mechanical attachment relief.

Preferably, the element in sheet form has a basis weight ranging from 20 to 300 g/m² and even more preferentially ranging from 30 to 200 g/m².

The element in sheet form notably has a thickness ranging from 40 to 1000 micrometres, preferably a thickness ranging from 40 to 400 micrometres and better still from 60 to 200 micrometres.

The element in sheet form may be opaque or transparent. Preferably, the element in sheet form is transparent, which facilitates its positioning on the hair, notably when it is desired to produce one or more patterns at a precise place on the lock or on the head of hair. In other words, the transparency of the element in sheet form facilitates the implementation of the dyeing process, notably in the production of coloured patterns, and improves its precision.

The element in sheet form used in the dyeing process according to the invention is preferably flexible and strong. Preferentially, the strength of the sheet is greater than 300 kPa (standard TAPPI-T403).

Preferably, the element in sheet form is water-resistant. In particular, the water absorption of said element is measured by the COBB 60 test which corresponds to the capacity of said element to absorb water during contact for 60 seconds (the procedure of which is given by the standard ISO 535, TAPPI-T411 measurement).

Thus, the element in sheet form absorbs less than 100 g/m² and preferentially less than 40 g/m² of water.

Preferably, the element in sheet form is resistant to oily compounds. Thus, use may be made of a “food” paper, i.e. a complex of paper and of polymeric compound of the polyethylene type or of paper and paraffin, which is capable of acting as a barrier to water and to oils.

The element in sheet form may optionally be covered with a deposit of an adhesive composition. This adhesive layer makes it possible to improve the adhesion of the oxidation dye(s) to the surface of the element in sheet form.

According to a preferred embodiment, the element in sheet form including a surface coated with at least one layer comprising one or more oxidation dyes may be covered with a protection means which serves to protect the surface of said element from external elements. Thus, the element in sheet form comprises on its surface one or more oxidation dyes that may be covered with a protective layer. Such a protective layer makes it possible to further minimize the impairment of the oxidation dye(s) caused by moisture, light or atmospheric oxygen.

Thus, the element in sheet form may be protected by implementing processes used in paper varnishing techniques (oil varnish, acrylic varnish, etc.), and in particular by using a water-based or organic acrylic varnish composition.

In this way, the surface of the element in sheet form can be protected with a layer of acrylic varnish.

In other words, the element in sheet form includes on its surface at least one layer containing one or more oxidation dyes and a layer of acrylic varnish; the layer of acrylic varnish being juxtaposed on the layer containing one or more oxidation dyes.

The mass per unit area of the layer of acrylic varnish ranges from 1 to 10 g/m² and more particularly from 2 to 5 g/m².

According to one variant, the element in sheet form is covered with a detachable protective sheet. For this purpose, the edges of the element in sheet form and of the protective sheet are bonded together by means of a fastening means, notably an adhesive, which may be produced via any type of method, notably by heat sealing. Thus, good cohesion is ensured between the protective sheet and the element in sheet form.

Advantageously, the protective sheet is UV-opaque to ensure better protection.

According to another variant, the element in sheet form may be covered with another protective means, namely a hermetic wrapping, defining above the element an oxygen-free space (under vacuum or under an inert atmosphere).

According to an advantageous embodiment, the element in sheet form is a plastic sheet covered with a thin layer of paper, in particular with a thickness of less than 50 μm and more preferentially less than 30 μm, such as cigarette paper or a layer of paper that can be broken down in the presence of water, such as toilet paper, or a thin layer of hydrophilic material such as cellulose or a hydrophilic silica, preferably having a thickness ranging from 5 to 200 μm.

In accordance with this embodiment, the layer of thin paper allows rapid drying and prevents the colouring from running following the application of a composition (B) as defined below. Furthermore, the layer of paper located below the thin paper absorbs little or none of the oxidation dye(s) derived from the element in sheet form, as a result of its low thickness. The colouring is thus satisfactorily rendered by the layer of thin paper on the keratin fibres, which notably leads to sharp coloured patterns. Furthermore, the element in sheet form in accordance with this embodiment makes it possible to minimize the dry areas under the keratin fibres.

When use is made of a support formed from a layer of paper, which is preferentially sparingly absorbent or non-absorbent, covered with a layer of paper that is capable of degrading on contact with water:

the layer of degradable paper (thickness possibly ranging from 10 to 200 μm) allows rapid drying and prevents the colouring from running following the application of composition (B) as defined below. Furthermore, the layer of paper located below the degradable paper absorbs little or none of the oxidation dye(s) derived from the element in sheet form, as a result of its low thickness. The colouring is thus satisfactorily rendered by the layer of thin paper on the keratin fibres, which notably leads to sharp coloured patterns. Furthermore, the element in sheet form in accordance with this embodiment makes it possible to minimize the dry areas under the keratin fibres.

In the case where a support formed from a layer of hydrophilic material is used:

the layer of hydrophilic material is typically from 5 to 200 μm thick, which allows rapid drying and prevents the colouring from running following the application of the oxidizing aqueous composition. This notably results in sharp coloured patterns.

According to a second advantageous embodiment, the element in sheet form is a microalveolar sheet, i.e. a sheet perforated with holes that are spaced apart from each other by a plastic material. Thus, composition (B) as defined below becomes housed in the holes of the substrate, which will make it possible to better render the power of the dyes on the keratin fibres after placing in contact with composition (B) as defined below.

The holes are located at the surface of the element in sheet form over a thickness ranging from 10% to 90% of the thickness of the sheet.

The oxidation dyes may be chosen from one or more couplers, optionally in combination with one or more oxidation bases.

In accordance with one embodiment, the oxidation dyes may be chosen from one or more couplers and one or more oxidation bases.

Preferably, the couplers are chosen from meta-phenylenediamines, meta-aminophenols, meta-diphenols, naphthalene-based couplers and heterocyclic couplers, and also the addition salts thereof and/or the solvates thereof.

Examples that may be mentioned include 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 5-methoxy-6-hydroxyindole, 2-amino-3-hydroxypyridine, 6-hydroxybenzomorpholine, 2-amino-4-hydroxyethylaminoanisole, 3-amino-6-methoxy-2-methylaminopyridine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 2-chloro-3,5-diaminopyridine, 2-chloro-3,5-diamino-6-methoxypyridine, 2-chloro-3,5-diamino-6-methylpyridine, 1-H-3-methylpyrazol-5-one, 1-phenyl-3-methylpyrazol-5-one, 4-(3,5-diaminopyridin-2-yl)-1-(2-hydroxyethyl)-1-methylpiperazin-1-ium chloride, 2,6-dimethylpyrazolo[1,5-b]-1,2,4-triazole, 2,4,6-trimethoxyaniline hydrochloride, 2,6-dimethyl[3,2-c]-1,2,4-triazole, 6-methylpyrazolo[1,5-a]benzimidazole and 2,6-diaminopyrazine, the addition salts thereof and/or the solvates thereof, and mixtures thereof.

Preferably, the coupler(s) used in the process of the invention are chosen from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-(β-hydroxyethylaminobenzene, 4-amino-2-hydroxytoluene, 5-amino-6-chloro-2-methylphenol, 2,4-diamino-1-(β-hydroxyethyloxy)benzene, α-naphthol, 6-hydroxyindole, 2-amino-3-hydroxypyridine, 6-hydroxybenzomorpholine, 3-amino-6-methoxy-2-methylaminopyridine and 2-amino-4-hydroxyethylaminoanisole, the addition salts thereof and/or the solvates thereof, and mixtures thereof.

Even more preferentially, the coupler(s) used in the process of the invention are chosen from 3-amino-6-methoxy-2-methylaminopyridine, 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene and 2-amino-4-hydroxyethylaminoanisole, the addition salts thereof and/or the solvates thereof, and mixtures thereof.

In general, the addition salts of the couplers that may be used within the context of the invention are notably chosen from the addition salts with an acid such as the hydrochlorides, hydrobromides, sulfates, citrates, succinates, tartrates, lactates, tosylates, benzenesulfonates, phosphates and acetates.

The oxidation bases may be chosen from para-phenylenediamines, bis(phenyl)alkylenediamines, para-aminophenols, ortho-aminophenols and heterocyclic bases, and the addition salts thereof.

Among the para-phenylenediamines, examples that may be mentioned include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols, examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols, examples that may be mentioned include 2-aminophenol, 2-amino-5-methylphenol, 2-amino-6-methylphenol, 5-acetamido-2-aminophenol and the addition salts thereof.

Among the heterocyclic bases, examples that may be mentioned include pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases that are useful in the present invention are the 3-aminopyrazolo[1,5-a]pyridine oxidation bases or the addition salts thereof described, for example, in patent application FR 2 801 308. Mention may be made, by way of example, of pyrazolo[1,5-a]pyrid-3-ylamine, 2-(acetylamino)pyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and the addition salts thereof.

Among the pyrimidine derivatives, mention may be made of the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof. Use may also be made of 4,5-diamino-1-(β-methoxyethyl)pyrazole.

Use will preferably be made of a 4,5-diaminopyrazole and even more preferentially of 4,5-diamino-1-(β-hydroxyethyl)pyrazole and/or a salt thereof.

Pyrazole derivatives that may also be mentioned include diamino-N,N-dihydropyrazolopyrazolones and notably those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a salt thereof.

As heterocyclic bases, use will preferentially be made of 4,5-diamino-1-(β-hydroxyethyl)pyrazole and/or 2,3-diamino-6,7-dihydro-1H,5H-pyrazolo[1,2-a]pyrazol-1-one and/or a salt thereof.

Preferably, the oxidation dyes are chosen from heterocyclic bases and heterocyclic or benzene-based couplers with at least one amine substituent on the aromatic ring.

Preferably, the oxidation dyes are chosen from couplers and more preferentially heterocyclic or benzene-based couplers with at least one amine substituent on the aromatic ring.

In accordance with the present invention, the substrate is pretreated with a dye composition, referred to as the dye preparation composition, comprising the oxidation dye(s).

Thus, the dye composition may cover all or part of the surface of the substrate. The surface of the substrate may thus be entirely or partially covered with one or more layers forming a dye composition.

Preferably, the dye preparation composition is deposited on part of the surface of the substrate so as to represent patterns, which, after contact with the keratin fibres, will make it possible to produce the coloured patterns on said fibres. In other words, the oxidation dye(s) are deposited in the form of patterns on the surface of the substrate. Thus, the surface of the substrate includes one or more layers consisting of the dye composition arranged in one or more particular geometrical forms, known as patterns, which, after reaction, lead to the production of coloured patterns on said fibres.

The pattern(s) may have any form, notably a geometrical form.

Preferably, the patterns may be squares, circles, ovals, ellipses or triangles, in the form of filled patterns or of lines surrounding these patterns. They may also be thick or thin, straight or curved lines, crossed lines, representing letters, stylized drawings or geometrical patterns. They may also be dotted lines or spots.

The substrate may include, on the face opposite the face bearing the oxidation dye(s), a copy of the desired pattern(s). The production of these patterns on the opposite face makes it possible to indicate the place where the oxidation dye(s) may then be deposited on the surface of the substrate. Such a production facilitates thereafter the positioning of the substrate on the keratin fibres at the place where it is desired to produce the pattern. In other words, the presence of the patterns on the opposite face makes it possible to indicate the place where the dye preparation composition may be deposited.

Preferably, the substrate is transparent.

According to one embodiment, the dyeing process according to the invention comprises the application to the keratin fibres of composition (A) as described previously, followed by the application of the substrate as defined previously to said fibres.

Composition (A) and the substrate may be used on the keratin fibres at room temperature, in particular at a temperature which may range from 23° C. to 33° C.

Composition (A) may be applied to the keratin fibres for a leave-on time which may range from 30 seconds to 2 hours.

Composition (A) may be applied using an applicator, in particular a brush, or by hand.

Preferably, on conclusion of the leave-on time of composition (A), the keratin fibres may be rinsed with water.

Preferably, the keratin fibres are not washed with surfactants, especially not washed with a shampoo.

According to a preferred embodiment, the process does not comprise the application to the keratin fibres of a washing composition comprising surfactants, especially a shampoo composition, between the application of a composition (A) containing one or more peroxygenated salts as previously defined and the substrate including a surface coated with at least one layer containing one or more oxidation dyes as previously defined.

Preferably, the substrate is then applied to the various areas of the keratin fibres to be treated.

Preferably, (ii) the application of the substrate is performed less than 3 hours after (i) the application of composition (A), preferably less than 1 hour, better still less than 45 minutes after (i) the application of composition (A).

In particular, the application of the substrate is performed less than 3 hours after the conclusion of the leave-on time of composition (A), preferably less than 1 hour, better still less than 45 minutes after the conclusion of the leave-on time of composition (A).

Advantageously, the leave-on time of the substrate on the keratin fibres may be less than 20 minutes, preferably last from 1 to 15 minutes, even preferably last from one to ten minutes.

More advantageously, the leave-on time of the substrate on the keratin fibres last from 1 to 10 minutes.

On conclusion of the leave-on time of the substrate, the keratin fibres are rinsed with water, optionally washed with a shampoo and then rinsed with water, before being dried or left to dry.

In accordance with this embodiment, the application of composition (A) to the keratin fibres may or may not lead to bleaching of said fibres before applying the substrate as defined previously.

In the case where the application of composition (A) to the keratin fibres leads to bleaching of the keratin fibres, the use of the substrate makes it possible to produce coloured patterns on all or a part of the area bleached with composition (A).

This variant is particularly suitable for keratin fibres which have a tone depth of less than 5.

In accordance with this variant, the substrate may be used on the keratin fibres after a time ranging from 30 seconds to 2 hours.

In accordance with this embodiment, the process according to the invention comprises:
the application to the keratin fibres of a composition resulting from the mixing of a composition (A) and of a composition comprising one or more chemical oxidizing agents, in particular hydrogen peroxide,
optional rinsing of the keratin fibres,
the application to said fibres of at least one substrate as described previously;

the process does not use any hydrogen peroxide during the application of the substrate to the fibres.

In particular, the process according to the invention comprises:
the mixing of composition (A) with a composition comprising one or more chemical oxidizing agents, in particular hydrogen peroxide,
the application to the keratin fibres of a composition resulting from the mixing of a composition (A) and of the composition comprising one or more chemical oxidizing agents, in particular hydrogen peroxide,
optional rinsing of the keratin fibres,
the application to said fibres of at least one substrate as described previously;

the process does not use any hydrogen peroxide during the application of the substrate to the fibres.

In the case where the application of composition (A) to the keratin fibres does not lead to bleaching of the keratin fibres, the use of the substrate makes it possible to produce coloured patterns on all or a part of the area treated with composition (A).

In accordance with this second variant, the substrate may be used on the keratin fibres after a time ranging from 30 seconds to 2 hours.

In accordance with this embodiment, the process according to the invention comprises:
the application of composition (A) to the keratin fibres,
optional rinsing of the keratin fibres,
the application to said fibres of at least one substrate as described previously;

the process does not use any hydrogen peroxide during the application of the substrate to the fibres.

According to another embodiment, the process according to the invention also comprises a step of heating the keratin fibres.

Preferably, the heating step makes it possible to heat to a temperature ranging from 60 to 250° C., preferably to a temperature ranging from 80° C. to 180° C., preferably ranging from 100° C. to 160° C. and better still from 120° C. to 150° C.

Preferably, the heating step is performed for a time which may range from 1 to 30 seconds and preferentially from 1 to 10 seconds.

In other words, the process according to the invention may successively comprise:
the application to the keratin fibres of a composition (A) as described previously,
the application to said fibres of at least one substrate as described previously,
heating of the keratin fibres.

Advantageously, the dyeing of the keratin fibres may be obtained after a few seconds.

According to one embodiment, the process according to the invention may also comprise the use of a composition (B) free of hydrogen peroxide after rinsing of the composition (A).

In particular, composition (B) is applied to the keratin fibres before or once these fibres have been placed in contact with the substrate so as to promote the contact between the substrate and composition (A).

Preferably, composition (B) is applied once the keratin fibres have been placed in contact with the substrate.

Composition (B) may comprise one or more organic solvents as described previously, preferably chosen from polyols containing more than two hydroxyl functions, such as glycerol.

Preferably, composition (B) may be aqueous and may comprise one or more organic solvents.

Composition (B) may also comprise one or more surfactants, preferably one or more nonionic and/or anionic surfactants.

Preferably, the nonionic surfactants are oxyalkylenated and chosen from oxyethylenated C₈-C₃₀ alcohols, and polyoxyethylenated esters of saturated or unsaturated, linear or branched C₈-C₃₀ acids and of sorbitol.

Composition (B) may also comprise one or more fatty substances.

The term “fatty substance” means an organic compound that is insoluble in water at ordinary temperature (25° C.) and at atmospheric pressure (760 mmHg) (solubility of less than 5%, preferably less than 1% and even more preferentially less than 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

According to the invention, the fatty substances are chosen from compounds that are liquid or pasty at room temperature and at atmospheric pressure.

More particularly, the fatty substance(s) are chosen from C₆-C₁₆ lower alkanes, non-silicone oils of animal, plant, mineral or synthetic origin, fatty alcohols, esters of a fatty acid and/or of a fatty alcohol, non-silicone waxes and silicones.

It is recalled that, for the purposes of the invention, fatty alcohols, esters and acids more particularly bear at least one linear or branched, saturated or unsaturated hydrocarbon-based group comprising 6 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

Preferably, the fatty substance(s) are chosen from fatty alcohols, notably those chosen from linear or branched, saturated or unsaturated alcohols including from 8 to 30 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

More preferentially, composition (B) comprises cetyl alcohol, stearyl alcohol and a mixture thereof, notably cetyl alcohol.

Compositions (A) and/or (B) may comprise one or more adjuvants such as anionic, cationic, nonionic, amphoteric or zwitterionic polymers or mixtures thereof, mineral thickeners, and in particular fillers such as clays or talc, organic thickeners/gelling agents with, in particular, anionic, cationic, nonionic and amphoteric polymeric associative thickeners other than the polymers mentioned previously, antioxidants, penetrants, sequestrants, fragrances, dispersants, film-forming agents, ceramides, preserving agents, opacifiers.

The adjuvants may be present for each of them in an amount ranging from 0.01% to 20% by weight relative to the total weight of the composition under consideration.

Preferably, the substrate is pretreated with at least one dye preparation composition comprising one or more oxidation dyes as defined previously.

The dye preparation composition may be liquid or in pulverulent form at room temperature, preferably liquid at room temperature.

The dye preparation composition preferably comprises one or more alkaline agents as defined previously.

When said dye preparation composition is aqueous and contains one or more alkaline agents, the pH of said composition preferably ranges from 7.5 to 13, better still from 8 to 12 and even better still from 8 to 11.

Preferably, the process for manufacturing the substrate as defined previously comprises at least one step of depositing onto the surface of said substrate at least one dye preparation composition containing one or more oxidation dyes and at least one step of drying said substrate; more preferentially, said dye preparation composition is deposited onto the surface of the substrate by means of a printing method.

In other words, the dye preparation composition(s) are printed on the surface of a substrate, that is to say using a printing process which makes it possible to obtain the substrate defined previously.

According to this preference, the printing method which serves to deposit the dye composition(s) onto the surface of the substrate may be a screen printing process, a flexography process, an offset printing process, an inkjet printing process or a laser printing process.

Preferentially, the dye preparation composition is printed onto the surface of the substrate by means of an inkjet printing process or a laser printing process, notably using an inkjet printer or a laser printer.

This preferred manufacturing process, corresponding to a process for printing the substrate, may be performed in the hairstyling salon itself, notably by means of the presence of an inkjet printer or a laser printer, before performing the dyeing process according to the invention.

Alternatively, this preferred process may also be performed outside the hairstyling salon and as such the user merely has to use the substrates to dye the hair.

In this case, the pretreated substrate may be supplied to the user to produce a unified colouring and/or patterns on the hair.

Preferably, the dye preparation composition containing the oxidation dye(s) is deposited in the form of one or more patterns onto the surface of a substrate. In particular, the patterns may be squares, circles, ovals, ellipses or triangles, in the form of filled patterns or of lines surrounding these patterns. They may also be thick or thin, straight or curved lines, crossed lines, representing letters, stylized drawings or geometrical patterns. They may also be dotted lines or spots.

Once the dye preparation composition(s) have been deposited at the surface of the substrate, the substrate is then dried.

The substrate thus pretreated in accordance with the manufacturing process preferably dries within a period ranging from 5 minutes to 120 minutes, preferentially from 5 minutes to 90 minutes, more preferentially from 1 minute to 60 minutes and better still from 5 minutes to 60 minutes.

Preferably, the step of drying said substrate consists in leaving said substrate to dry in the open air.

Once the substrate has been prepared, it comprises a surface coated with at least one layer comprising one or more oxidation dyes as defined above.

The layer coating the surface of the substrate is a dye composition comprising one or more oxidation dyes as defined above.

In accordance with the present invention, the layer preferably comprises a total water content of less than 20% by weight, preferably less than or equal to 15% by weight, more preferentially less than or equal to 10% by weight, relative to the total weight of the dye composition.

The examples that follow serve to illustrate the present invention without, however, being limiting in nature.

### EXAMPLES

In the examples that follow, all the amounts are indicated as weight percentages of active material relative to the total weight of the composition.

### Example 1

a) Test Compositions

The following compositions were prepared from the ingredients whose contents are indicated in the tables below:

TABLE 1

| | Composition A1 |
|---|---|
| Pigment (ultramarine blue) | 0.25 |
| Magnesium carbonate hydroxide | 9.4 |
| Sodium silicate | 18.3 |
| Disodium EDTA | 0.99 |
| L-Proline | 0.05 |
| Sodium metasilicate | 2.48 |
| Sodium lauryl sulfate | 0.99 |
| Threonine | 0.05 |
| Potassium persulfate | 48.33 |
| Ammonium persulfate | 4.9 |
| Mineral oil | 2 |
| Sodium stearate | 9.87 |
| Guar gum | 2.5 |
| Water | qs 100 |

TABLE 2

| | Composition A2 |
|---|---|
| Trideceth-2 monoethanolamine carboxamide | 0.85 |
| Sodium stannate | 0.04 |
| Pentasodium pentetate | 0.06 |
| Glycerol | 0.5 |
| Stearyl alcohol | 2.28 |
| Ceteareth-25 | 0.57 |
| Phosphoric acid | qs |
| Hydrogen peroxide | 12 |
| Tetrasodium pyrophosphate | 0.02 |
| Water | qs 100 |

TABLE 3

| | Composition C |
|---|---|
| Trideceth-2 monoethanolamine carboxamide | 0.85 |
| Tetrasodium etidronate | 0.06 |
| Sodium salicylate | 0.035 |
| Glycerol | 0.5 |
| Stearyl alcohol | 2.28 |
| Ceteareth-25 | 0.57 |
| Tetrasodium pyrophosphate | 0.02 |
| Water | qs 100 |

TABLE 4

| | Dye preparation |
|---|---|
| | composition 1 (P1) |
| Propylene glycol | 6.2 |
| Hexylene glycol | 3 |
| Dipropylene glycol | 3 |
| Sodium hydroxide | qs |
| Phosphoric acid | qs |
| 2,3-Diaminodihydropyrazolopyrazolone | 2.5 |
| dimethosulfonate | |
| 2,4-Diaminophenoxyethanol hydrochloride | 1.74 |
| Ascorbic acid | 0.25 |
| Sodium metabisulfite | 0.25 |
| Denatured alcohol | 8.8 |
| Water | qs 100 |

b) Procedure

b1) Preparation of the Substrate

The dye preparation composition (P1) is applied uniformly to a substrate S1 of paper type using a HandCoater (sold by the company RK Print Coat Instruments Ltd., K-HandCoater range, under the yellow reference). The deposit of the dye preparation composition is 5 mg/cm².

b2) Implementation of the Process

Compositions (A1) and (A2) are mixed weight for weight to obtain a composition (A) based on persulfates.

Composition (A) is applied to a lock of TD6 hair at a temperature of 33° C., at a rate of 10 g of composition per 1 g of lock of hair.

On conclusion of a leave-on time lasting 50 minutes, the lock is rinsed with water.

Substrate S1 prepared in accordance with step b1) is then applied to the bleached lock. The lock of hair is impregnated using composition (C), at a rate of 3 g of composition per 1 g of lock, so as to allow good contact between the sheet and the lock.

On conclusion of a leave-on time lasting 5 minutes, the lock is rinsed with water and washed with a standard shampoo.

The substrate is applied, on the one hand, to a lock of hair which has just been bleached with composition (A) (freshly bleached lock) and, on the other hand, to a lock of hair which was bleached with composition (A) one week previously.

c) Results

The difference in colour ΔE of the lock of hair which was treated by means of the process according to the invention, in accordance with step b2), relative to the lock of hair bleached after the application of composition (A), is evaluated.

The difference in colour ΔE is determined using a Minolta CM 2002 colorimeter in the CIE L* a* b* international system.

ΔE corresponds to the difference in colour between a lock of bleached hair and a lock of unbleached hair, i.e. of hair not treated with composition (A), and is measured according to the following equation:

ΔE=\sqrt{(L*−Lₒ*)²+(a*−aₒ*)²+(b*−bₒ*)²} (i)

In this equation, L*, a* and b* represent, respectively, the values measured on the lock bleached following the implementation of the process according to the invention, and L₀*, a₀* and b₀* represent, respectively, the values measured on the lock bleached with composition (A).

In this L* a* b* system, L* represents the lightness, a* indicates the green/red colour axis and b* indicates the blue/yellow colour axis. The higher the value of L, the lighter or less intense the colour. Conversely, the lower the value of L, the darker or more intense the colour. The higher the value of a*, the redder the shade, and the higher the value of b*, the yellower the shade.

The results are collated in the table below:

TABLE 5

| | ΔE |
|---|---|
| Freshly bleached lock | 52.33 |
| Lock bleached more than one week | 12.05 |
| previously | |

It is observed that the implementation of the process according to the invention leads to satisfactory colouring with a leave-on time of the substrate lasting 5 minutes.

### Example 2

a) Test Compositions

TABLE 6

| | Dye preparation |
|---|---|
| | composition 2 (P2) |
| Propylene glycol | 6.2 |
| Hexylene glycol | 3 |
| Dipropylene glycol | 3 |
| Sodium hydroxide | qs |
| Phosphoric acid | qs |
| 2,4-Diaminophenoxyethanol hydrochloride | 2.5 |
| Ascorbic acid | 0.25 |
| Sodium metabisulfite | 0.25 |
| Denatured alcohol | 8.8 |
| Water | qs 100 |

b) Procedure

b1) Preparation of the Substrate

The dye preparation composition (P2) is applied uniformly to a substrate S2 of paper type using a HandCoater (sold by the company RK Print Coat Instruments Ltd., K-HandCoater range, under the yellow reference). The deposit of the dye preparation composition is 5 mg/cm².

b2) Implementation of the Process

Compositions (A1) and (A2) are mixed weight for weight to obtain a composition (A) based on persulfates.

Composition (A) is applied to a lock of TD6 hair at a temperature of 33° C., at a rate of 10 g of composition per 1 g of lock of hair.

On conclusion of a leave-on time lasting 50 minutes, the lock is rinsed with water.

Substrate S2 prepared in accordance with step bl) is then applied to the bleached lock. The lock of hair is impregnated using composition (C), at a rate of 3 g of composition per 1 g of lock, so as to allow good contact between the sheet and the lock.

On conclusion of a leave-on time lasting 5 minutes, the lock is rinsed with water and washed with a standard shampoo.

The substrate is applied, on the one hand, to a lock of hair which has just been bleached with composition (A) (freshly bleached lock) and, on the other hand, to a lock of hair which was bleached with composition (A) one week previously.

c) Results

The difference in colour ΔE of the lock of hair which was treated by means of the process according to the invention, in accordance with step b2), relative to the lock of hair bleached after the application of composition (A), is evaluated.

The difference in colour ΔE is determined using a Minolta CM 2002 colorimeter in the CIE L* a* b* international system.

ΔE corresponds to the difference in colour between a lock of bleached hair and a lock of unbleached hair, i.e. of hair not treated with composition (A), and is measured according to the following equation:

ΔE=\sqrt{(L*−Lₒ*)²+(a*−aₒ*)²+(b*−bₒ*)²} (i)

In this equation, L*, a* and b* represent, respectively, the values measured on the lock bleached following the implementation of the process according to the invention, and L₀*, a₀* and b₀* represent, respectively, the values measured on the lock bleached with composition (A).

In this L* a* b* system, L* represents the lightness, a* indicates the green/red colour axis and b* indicates the blue/yellow colour axis. The higher the value of L, the lighter or less intense the colour. Conversely, the lower the value of L, the darker or more intense the colour. The higher the value of a*, the redder the shade, and the higher the value of b*, the yellower the shade.

The results are collated in the table below:

TABLE 7

| | ΔE |
|---|---|
| Freshly bleached lock | 30.25 |
| Lock bleached more than one week | 1.49 |
| previously | |

### Example 5

a) Test Compositions

An acidic sodium persulfate solution A3 (10% of sodium persulfate at pH 2.4 with phosphoric acid) is prepared.

b) Procedure

Solution (A3) is applied to locks of TD8 hair for a time of 10 minutes at a temperature of 27° C., at a rate of 5 g of composition (A3) per 1 g of lock. The lock is then rinsed.

The lock of hair is positioned on the substrate S1. After it has been positioned, the lock is impregnated with composition (C) so as to promote good contact between the sheet and the lock, at a rate of 3 g of composition (C) per 1 g of hair. On conclusion of a leave-on time of 10 minutes, the lock is rinsed with water and washed with a standard shampoo.

This process is compared with a process in which water is applied instead of the solution (A3).

c) Results

The difference in colour ΔE of the lock of hair which was treated by means of the process according to the invention relative to the unbleached lock of hair is evaluated.

The difference in colour ΔE is determined using a Minolta CM 2002 colorimeter in the CIE L* a* b* international system and is measured according to equation (i) described previously in which L*, a* and b* represent, respectively, the values measured on the lock bleached following the implementation of the process according to the invention and L₀*, a₀* and b₀* represent, respectively, the values measured on the unbleached block.

c1) Test of Not Bleaching the Lock with (A3)

It is first checked that the application of the solution (A3) to locks of hair does not result in them being bleached.

To do this, solution (A3) is applied to a lock of TD4 hair for a time of 15 minutes at a temperature of 27° C., at a rate of 5 g of solution (A3) per 1 g of lock.

The results are collated in the following table:

TABLE 8

| | ΔL | ΔE |
|---|---|---|
| TD4 lock treated with solution A3 | 1.23 | 1.24 |

It is found that the application of solution (A3) to the lock of TD4 hair leads to a difference in colour ΔE of less than 2, which means that the lock of hair is not bleached.

c2) Results of the Process

TABLE 9

| State of the lock | Substrate | ΔE |
|---|---|---|
| Lock treated with solution (A3) | S1 | 8.92 |
| Lock treated with water | | 1.90 |

It is found that the process according to the invention makes it possible to result in higher colour build-ups than a process in which water is used with a substrate S1.

1. Process for dyeing keratin fibres, in particular human keratin fibres such as the hair, successively applying to said fibres:
(i) a composition (A) containing one or more peroxygenated salts,
(ii) a substrate including a surface coated with at least one layer containing one or more oxidation dyes,
given that the process does not use any hydrogen peroxide during the application of the substrate to the fibres.

2. Process according to claim 1, characterized in that it comprises a rinsing step between (i) the application of composition (A) and (ii) the application of the substrate.

3. Process according to claim 1, characterized in that (ii) the application of the substrate is performed less than 3 hours after the conclusion of the leave-on time of composition (A), preferably less than 1 hour, better still less than 45 minutes after the conclusion of the leave-on time of composition (A).

4. Process according to claim 1, characterized in that the peroxygenated salt(s) are chosen from persulfates, perborates, peracids and/or salts thereof, percarbonates, in particular of alkali metals or alkaline-earth metals, and mixtures thereof, preferably from persulfates.

5. Process according to claim 1, characterized in that the oxidation dyes are chosen from couplers and optionally oxidation bases, preferably couplers.

6. Process according to claim 5, characterized in that the couplers are chosen from meta-phenylenediamines, meta-aminophenols, meta-diphenols, naphthalene-based couplers and heterocyclic couplers, and also the addition salts thereof and/or the solvates thereof.

7. Process according to claim 5, characterized in that the couplers are chosen from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 5-methoxy-6-hydroxyindole, 2-amino-3-hydroxypyridine, 6-hydroxybenzomorpholine, 2-amino-4-hydroxyethylaminoanisole, 3-amino-6-methoxy-2-methylaminopyridine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 2-chloro-3,5-diaminopyridine, 2-chloro-3,5-diamino-6-methoxypyridine, 2-chloro-3,5-diamino-6-methylpyridine, 1-H-3-methylpyrazol-5-one, 1-phenyl-3-methylpyrazol-5-one, 4-(3,5-diaminopyridin-2-yl)-1-(2-hydroxyethyl)-1-methylpiperazin-1-ium chloride, 2,6-dimethylpyrazolo[1,5-b]-1,2,4-triazole, 2,4,6-trimethoxyaniline hydrochloride, 2,6-dimethyl[3,2-c]-1,2,4-triazole, 6-methylpyrazolo[1,5-a]benzimidazole, 2,6-diaminopyrazine, the addition salts thereof and/or the solvates thereof, and mixtures thereof, preferably from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, α-naphthol, 6-hydroxyindole, 2-amino-3-hydroxypyridine, 6-hydroxybenzomorpholine, 3-amino-6-methoxy-2-methylaminopyridine, 2-amino-4-hydroxyethylaminoanisole, the addition salts thereof and/or the solvates thereof, better still from 3-amino-6-methoxy-2-methylaminopyridine, 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene, 2-amino-4-hydroxyethylaminoanisole, the addition salts thereof and/or the solvates thereof, and mixtures thereof.

8. Process according to claim 1, characterized in that it also comprises a step of heating the keratin fibres.

9. Process according to claim 8, characterized in that the heating step raises the temperature of the keratin fibres to a temperature ranging from 60 to 250° C., preferably to a temperature ranging from 80° C. to 180° C., preferably ranging from 100° C. to 160° C. and better still from 120° C. to 150° C.

10. Process according to claim 1, characterized in that the leave-on time of the substrate on the keratin fibres is less than 20 minutes, preferably last from 1 to 15 minutes, even preferably last from 1 to 10 minutes.

11. Process according to claim 1, characterized in that the substrate is an element in sheet form.

12. Process according to claim 11, characterized in that the element in sheet form is made of plastic material, in particular thermoplastic, paper, metal, notably aluminium, a woven, a nonwoven of non-absorbent fibres, notably of cellulose or a derivative thereof, or polyamide 6,6.

13. Process according to claim 11, characterized in that the element in sheet form includes an adhesive layer on which is deposited the layer containing at least said oxidation dye.

14. Process according to claim 1, characterized in that it also comprises the use of a composition (B) after using the substrate.

15. Process according to claim 14, characterized in that composition (B) comprises one or more organic solvents.
